(19) United States
(12) Patent Application Publication
(10) Pub. No.: US 2007/0276153 A1
(43) Pub. Date: Nov. 29, 2007

(54) Diastereoselective Method of Preparing Olefins by Means of the Horner-Wadsworth-Emmons Reaction Using a Particular Phosphonate Which Improves Diastereoselectivity at all Temperatures Including at Ambient Temperature

(75) Inventors: François Touchard, Villeurbanne (FR); Laurent Saint-Jalmes, Vourles (FR)

Correspondence Address: Jean-Louis Seugnet;Rhodia INC., 8 Cedar Brook Drive, CN 7500, Cranbury, NJ, 08512-7500, US

(73) Assignee: RHODIA UK LIMITED, HERTFORDSHIRE WD24 4QP, UNITED KINGDOM (GB)

(21) Appl. No.: 10/578,396

(22) PCT Filed: Nov. 4, 2004
(86) PCT No.: PCT/FR04/02834

§ 371 Date: Jan. 25, 2007

(30) Foreign Application Priority Data
Nov. 4, 2003 (FR) 0312921

(51) Int. Cl. C07C 67/313

(52) U.S. Cl. 560/114; 560/206

(57) ABSTRACT

The invention relates to a diastereoselective process for the preparation of olefins by the Horner-Wadsworth-Emmons reaction which consists in reacting a specific phosphonate improve the diastereoselectivity at all temperatures including at ambient temperature, with a carbonyl derivative in the presence of a base in an appropriate solvent.

The present invention relates to a diastereoselective process for the preparation of olefins by the Horner-Wadsworth-Emmons reaction which consists in reacting a phosphonate with a carbonyl derivative in the presence of a base in an appropriate solvent.
The reaction involved is as follows:

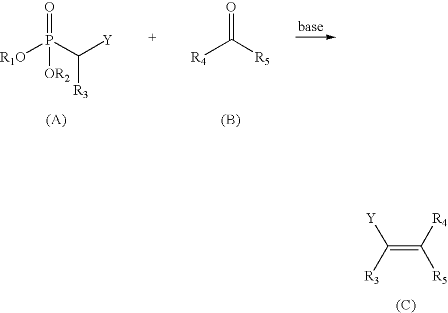

The carbonyl compound (B) can be an aldehyde or a ketone, with the condition that R₄ has priority over R₅ according to the Cahn-Ingold-Prelog rules. The latter are described, for example, in the book entitled “Advanced Organic Chemistry: Reactions, Mechanisms, and Structure”, third edition, Jerry March, John Wiley & sons, 1985, the content of pages 96 to 112 of which is incorporated by reference.

The Applicant Company has just discovered that, unexpectedly, the use of specific phosphonates makes it possible to improve the diastereoselectivity in the Horner-Wadsworth-Emmons reaction, this being the case whatever the temperature.

Thus, a subject matter of the present invention is a process for the diastereoselective preparation of olefins (C) by the Horner-Wadsworth-Emmons reaction which consists in reacting a phosphonate (A) with a carbonyl derivative (B) in the presence of a base in an appropriate solvent, 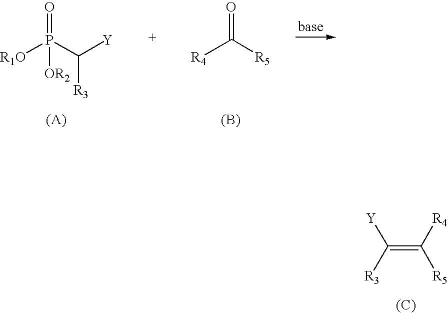
in which the compounds (A), (B) and (C) are such that:

Y represents an electron-withdrawing group known to a person skilled in the art and chosen so as not to interfere with the Horner-Wadsworth-Emmons reaction. Mention may in particular by made, among these groups, of:
CO₂R,
CN,
C(O)R,
S(O)R,
S(O)₂R,
C(O)NRR′,
N═CRR′,
P(O)OROR′,
with R and R′ as defined below,

R₅, R and R′, taken independently, can be identical or different and they represent:
a hydrogen atom;
a saturated or unsaturated and linear or branched aliphatic radical having from 1 to 24 carbon atoms which is optionally substituted by heteroatoms;
a saturated, unsaturated or aromatic and monocyclic or polycyclic cycloaliphatic radical having from 4 to 24 carbon atoms which is optionally substituted by heteroatoms;
a saturated or unsaturated and linear or branched aliphatic radical carrying a cyclic substituent which is optionally substituted by heteroatoms in the aliphatic part and/or the cyclic part;

R and R′ can also be taken together to form a saturated, unsaturated or aromatic ring optionally comprising heteroatoms;

R₃ represents a radical chosen from:
R,
a halogen atom,
OR,
SR,
NRR′,
with R and R′ as defined above,

R₄ represents a radical chosen from:
a saturated or unsaturated and linear or branched aliphatic radical having from 1 to 24 carbon atoms which is optionally substituted by heteroatoms;
a saturated, unsaturated or aromatic and monocyclic or polycyclic cycloaliphatic radical having from 4 to 24 carbon atoms which is optionally substituted by heteroatoms; it being possible for the heteroatoms also to be present in the cyclic part;
a saturated or unsaturated and linear or branched aliphatic radical carrying a cyclic substituent which is optionally substituted by heteroatoms in the aliphatic part and/or the cyclic part;

with the condition that R₄ has priority over R₅ according to the Cahn-Ingold-Prelog rules,

characterized in that R₁ and R₂, taken independently, can be identical or different and they represent a radical of formula (I): 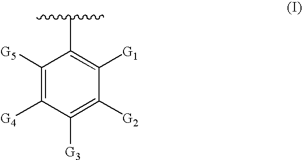
in which:

G₁, G₂, G₃, G₄ and G₅, taken independently, can be identical or different and they represent:
a hydrogen atom,
an alkyl radical having from 1 to 24 carbon atoms and preferably 1 to 12 carbon atoms and more preferably still 1 to 6 carbon atoms, which can be:
   a saturated or unsaturated and linear or branched aliphatic radical which is optionally substituted by heteroatoms; such as, for example, a carbon atom bonded to three carbon atoms, and preferably tert-butyl;
   a saturated, unsaturated or aromatic and monocyclic or polycyclic cycloaliphatic radical having from 4 to 24 carbon atoms which is optionally substituted by:
      an alkoxy radical having from 1 to 24 carbon atoms,
      a halogen atom,
      a heteroatom, such as an oxygen atom, a sulfur atom or a nitrogen atom, it being possible for the heteroatom also to be present in the cyclic part;
   a saturated or unsaturated and linear or branched aliphatic radical carrying a cyclic substituent which is optionally substituted by heteroatoms in the aliphatic part and/or the cyclic part;
an alkoxy radical having from 1 to 24 carbon atoms,
a halogen atom,
a heteroatom, such as an oxygen atom, a sulfur atom or a nitrogen atom,

G₁, G₂, G₃, G₄ or G₅ can also be taken together to form, between two neighboring groups, a saturated, unsaturated or aromatic ring having from 4 to 6 carbon atoms and optionally comprising heteroatoms,

it being understood that at least one of the G₁ or G₅ radicals is taken independently and represents a radical formed by a carbon atom itself connected to three carbon atoms, and preferably a tert-butyl radical, or a phenyl radical optionally substituted by one or more radicals chosen from alkoxy radicals having from 1 to 24 carbon atoms, halogen atoms or heteroatoms, such as an oxygen atom, a sulfur atom or a nitrogen atom.

Use is preferably made of a phosphonate (A) in which R₁ and R₂, which are identical or different, have the formula (I) in which at least one of the G₁ or G₅ radicals is taken independently and represents a radical formed by a carbon atom itself connected to three carbon atoms, and preferably a tert-butyl radical.

Phosphonates which are particularly advantageous in the context of the invention are phosphonates of formula (A) in which R₁ is identical to R₂ and has the formula (I) in which:

G₁ is tert-butyl and G₂, G₃, G₄ and G₅ are hydrogen atoms,

G₁ and G₃ are tert-butyl radicals and G₂, G₄ and G₅ are hydrogen atoms, or

G₁ is a phenyl radical and G₂, G₃, G₄ and G₅ are hydrogen atoms.

Among these advantageous phosphonates, the phosphonate used for the reaction can be chosen from the phosphonates of formula (A) in which:

R₁ is identical to R₂ and has the formula (I) in which:

G₁ is tert-butyl and G₂, G₃, G₄ and G₅ are hydrogen atoms,

G₁ and G₃ are tert-butyl radicals and G₂, G₄ and G₅ are hydrogen atoms, or

G₁ is a phenyl radical and G₂, G₃, G₄ and G₅ are hydrogen atoms,

and Y represents CO₂R, with R representing a hydrogen atom or a saturated or unsaturated and linear, branched or cyclic alkyl radical having from 1 to 12 carbon atoms,

and R₃ represents a hydrogen atom.

Use is preferably made of a phosphonate of formula (A) in which:

R₁ is identical to R₂ and has the formula (I) in which:

G₁ is tert-butyl and G₂, G₃, G₄ and G₅ are hydrogen atoms,

G₁ and G₃ are tert-butyl radicals and G₂, G₄ and G₅ are hydrogen atoms, or

G₁ is a phenyl radical and G₂, G₃, G₄ and G₅ are hydrogen atoms,

and Y represents a CO₂R radical, with R representing an ethyl radical;

and R₃ represents a hydrogen atom.

The carbonyl derivative (B) used for the reaction can be an aldehyde or a ketone. The R₄ and R₅ substituents are, of course, chosen so as not to interfere with the Horner-Wadsworth-Emmons reaction. One condition according to the Cahn-Ingold-Prélog rule has been imposed, so as to define the stereochemistry of the olefin preferably obtained (C). The Cahn-Ingold-Prélog rule is described, for example, in the book entitled “Advanced Organic Chemistry: Reactions, Mechanisms, and Structure”, third edition, Jerry March, John Wiley & sons, 1985, the content of pages 96 to 112 of which is incorporated by reference.

The carbonyl derivative (B) is preferably chosen from aldehydes, which corresponds to R₅ representing a hydrogen atom. The aldehydes used can, depending on the nature of the R₄ radical, be aliphatic and can optionally comprise ethylenic unsaturations, or they can be aromatic. In the case where the aldehydes used are aromatic, they can comprise optional substitutions by electron-donating or electron-withdrawing groups.

Mention may be made, as electron-donating groups, of C₁-C₆-alkyl, C₁-C₆-alkoxy, SR, NRR′ or phenyl groups, the phenyl group being, if appropriate, substituted by an alkyl or alkoxy group as defined above.

Within the meaning of the present invention, the term “electron-withdrawing group” is understood to mean a group as defined by H. C. Brown in the book entitled “Advanced Organic Chemistry: Reactions, Mechanisms, and Structure”, third edition, Jerry March, John Wiley & sons, 1985, the content of pages 243 and 244 of which is incorporated by reference. Mention may in particular be made, by representation of the electron-withdrawing groups, of:
a halogen atom,
an SO₂R group with R as defined above,
a CN or NO₂ group.

Mention may be made, among aliphatic aldehydes, of cyclohexanecarboxaldehyde (R₄ is a cyclohexyl radical) or an aliphatic aldehyde in which R₄ is n-C₇H₁₅. Mention may be made, among aromatic aldehydes, of benzaldehyde (R₄ represents a phenyl radical) or an aldehyde characterized in that the R₄ radical used is aromatic and optionally comprises one or more substitutions by (donating or withdrawing) alkoxy groups having from 1 to 6 carbon atoms or halogen atoms.

Thus, the aromatic aldehyde can comprise heteroatoms in the aromatic ring.

The aromatic aldehyde can also comprise substitutions by CF₃ groups.

The base is chosen from:

amides of MNR″R′″ type with M an alkali metal, such as lithium, sodium or potassium, and R″ and R′″ being chosen from alkyl radicals or radicals of alkylsilane type, such as the sodium or potassium salts of hexamethyldisilazane (NaHMDS or KHMDS),

alkoxides of MOR″ type with M an alkali metal, such as lithium, sodium or potassium, and R″ being chosen from alkyl radicals, such as potassium tert-butoxide (tBuOK),

hydrides of MH type with M an alkali metal, such as lithium, sodium or potassium,

carbonates of M₂CO₃ or MCO₃ type with M an alkali metal, such as lithium, sodium, potassium or cesium, or an alkaline earth metal, such as calcium or barium,

alkali metal or alkaline earth metal hydroxides, such as LiOH, NaOH, KOH, CsOH, Mg(OH)₂, Ca(OH)₂ or Ba(OH)₂,

alkali metal or alkaline earth metal phosphates, such as Li₃PO₄, Na₃PO₄, K₃PO₄, Cs₃PO₄ or Mg₃(PO₄)₂, or

organic nitrogenous bases of amine, amidine or guanidine type, such as, for example, 1,8-diazabicyclo[5.4.0]undec-7-ene (DBU) or 1,1,3,3-tetramethylguanidine (TMG), optionally in combination with alkali metal or alkaline earth metal halides.

Use is preferably made of a base chosen from:

alkoxides of MOR″ type with M an alkali metal, such as lithium, sodium or potassium, and R″ being chosen from alkyl radicals, such as potassium tert-butoxide (tBuOK),

carbonates of M₂CO₃ or MCO₃ type with M an alkali metal, such as lithium, sodium, potassium or cesium, or an alkaline earth metal, such as calcium or barium,

alkali metal or alkaline earth metal hydroxides, such as LiOH, NaOH, KOH, CsOH, Mg(OH)₂, Ca(OH)₂ or Ba(OH)₂,

alkali metal or alkaline earth metal phosphates, such as Li₃PO₄, Na₃PO₄, K₃PO₄, Cs₃PO₄ or Mg₃(PO₄)₂, or

organic nitrogenous bases of amine, amidine or guanidine type, such as, for example, 1,8-diazabicyclo[5.4.0]undec-7-ene (DBU) or 1,1,3,3-tetramethylguanidine (TMG), optionally in combination with alkali metal or alkaline earth metal halides.

More preferably still, use is made of a base chosen from:

carbonates of M₂CO₃ or MCO₃ type with M an alkali metal, such as lithium, sodium, potassium or cesium, or an alkaline earth metal, such as calcium or barium,

alkali metal or alkaline earth metal hydroxides, such as LiOH, NaOH, KOH, CsOH, Mg(OH)₂, Ca(OH)₂ or Ba(OH)₂, or

alkali metal or alkaline earth metal phosphates, such as Li₃PO₄, Na₃PO₄, K₃PO₄, Cs₃PO₄ or Mg₃(PO₄)₂.

The solvent used can be chosen from:
ethers, and preferably cyclic ethers, such as tetrahydrofuran (THF) or dioxane,
nitriles having from 1 to 8 carbon atoms, such as, for example, acetonitrile, methylglutaronitrile (MGN), adiponitrile (ADN) or benzonitrile, with a preference for acetonitrile, or
polar solvents of amide type, such as, for example, dimethylformamide (DMF), N-methylpyrrolidone (NMP) or dimethylacetamide (DMAC).

The amount of solvent used is generally between 0.5 ml and 20 ml per mmol of phosphonate.

The improvement in the selectivity of the reaction in the presence of the phosphonate of the invention and under the conditions of implementation of the invention, that is to say in the presence of carefully chosen bases and solvents, is observed whatever the temperature. It is thus possible to carry out the process of the invention at low temperature but it is also possible to carry it out at a temperature of 0° C. or at ambient temperature, that is to say approximately 25° C., while retaining a high diastereoselectivity.

This effect is surprising as this was not the case with the phosphonates used previously in the Horner-Wadsworth-Emmons reaction.

This effect is particularly advantageous from the viewpoint of industrial operation.

It makes it possible to carry out the process at a temperature of 0° C. or approximately 25° C. while retaining a high diastereoselectivity for olefin (C).

The process according to the invention can thus be carried out at a temperature of between −100° C. and +100° C.

Preferably, the process according to the invention is carried out at a temperature of between −50° C. and +50° C.

More preferably still, the process according to the invention is carried out at a temperature of between −20° C. and +50° C., indeed even at a temperature of between −10° C. and +25° C.

Other aspects and advantages of the processes which are subject matters of the invention will become apparent in the light of the examples which are presented below, by way of illustration and without implied limitation.

### EXAMPLE A

### Examples of the Synthesis of Phosphonates of the Invention

### Example A1

Synthesis of the Phosphonate I

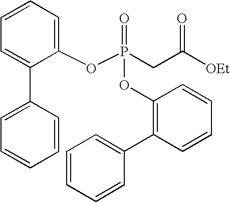

22.4 g (0.130 mol) of 2-phenylphenol and 14 g (0.137 mol) of triethylamine are dissolved in 100 ml of toluene and the mixture is cooled to 0° C. A solution of 10 g (0.067 mol) of PCl₂(OEt) in 40 ml of ether is then added so as to keep the temperature below 5° C. After 30 minutes at 0° C., the mixture is stirred at ambient temperature for an additional three hours. The salts are then filtered off and washed with toluene. The organic phase is subsequently treated over basic alumina in order to remove possible phosphorus-comprising byproducts. Finally, the solvent is evaporated to result in 25.4 g of mixed phosphite. 19.8 g (48.0 mmol) of this phosphite are subsequently added over 1 h to 12.3 g (72.4 mmol) of ethyl bromoacetate at 120° C. After reacting for 20 h, the excess ethyl bromoacetate is removed under vacuum to result in 20 g of phosphonate.

¹H NMR: 1.00(t, J=7.15 Hz, 3H), 2.41 (d, J=21.7 Hz, 2H), 3.89(q, J=7.15 Hz, 2H), 7.18-7.27 (m, 18H)

³¹P NMR: 12.7 ppm

¹³C NMR:13.8(s, CH₃), 34.1 (d, J=138.6 Hz, PCH₂), 61.6 (s, CH₂), 121.3 (d, J=2.7 Hz, 2CHₐᵣₒₘ), 125.5 (d, J=1.0 Hz, 2CHₐᵣₒₘ), 127.3 (s, 2CHₐᵣₒₘ), 128.1 (s, 4CHₐᵣₒₘ), 128.6 (d, J=1.3 Hz, 2Cₐᵣₒₘ), 129.3 (s, 4CHₐᵣₒₘ), 131.1 (s, 2CHₐᵣₒₘ), 133.6 (d, J=5.9 Hz, 2Cₐᵣₒₘ), 137.1 (s, 2Cₐᵣₒₘ), 147.1 (d, J=8.9 Hz, 2Cₐᵣₒₘ), 164.2 (d, J=6.2 Hz, C═O)

### Example A2

Synthesis of the Phosphonate II

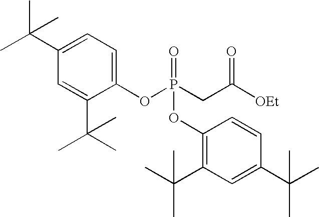

27.1 g (0.130 mol) of 2,4-di(tert-butyl)phenol and 14 g (0.137 mol) of triethylamine are dissolved in 100 ml of toluene and the mixture is cooled to 0° C. A solution of 10 g (0.067 mol) of PCl₂(OEt) in 40 ml of ether is then added so as to keep the temperature below 5° C. After 30 minutes at 0° C., the mixture is stirred at ambient temperature for an additional three hours. The salts are then filtered off and washed with toluene. The organic phase is subsequently treated over basic alumina in order to remove possible phosphorus-comprising byproducts. Finally, the solvent is evaporated to result in 30.4 g of mixed phosphite. The 30.4 g (63 mmol) of phosphite are subsequently added over 1 h to 16.2 g (95 mmol) of ethyl bromoacetate at 120° C. After reacting for 50 h, the excess ethyl bromoacetate is removed under vacuum to result in 32 g of phosphonate.

¹H NMR: 1.07 (t, J=7.15 Hz, 3H), 1.22 (s, 18H), 1.32 (s, 18H), 3.26 (d, J=21.4 Hz, 2H), 4.04 (q, J=7.15 Hz, 2H), 7.07 (dd, J=8.8 Hz, J=2.4 Hz, 2H), 7.30 (t, J=2.2 Hz, 2H), 7.49 (dd, J=8.5 Hz, J=1.1 Hz, 2H)

³¹P NMR: 10.3ppm

### Example A3

First Route For the Synthesis of the Phosphonate III

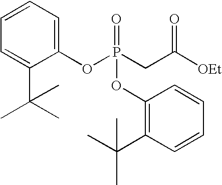

19.7 g (0.130 mol) of 2-(tert-butyl)phenol and 14 g (0.137 mol) of triethylamine are dissolved in 100 ml of toluene and the mixture is cooled to 0° C. A solution of 10 g (0.067 mol) of PCl₂(OEt) in 40 ml of ether is then added so as to keep the temperature below 5° C. After 30 minutes at 0° C., the mixture is stirred at ambient temperature for an additional three hours. The salts are then filtered off and washed with toluene. The organic phase is subsequently treated over basic alumina in order to remove possible phosphorus-comprising byproducts. Finally, the solvent is evaporated to result in 23.3 g of mixed phosphite. 20 g (53 mmol) of this phosphite are subsequently added over 1 h to 16.3 g (106 mmol) of ethyl bromoacetate at 130° C. After reacting for 20 h, the excess ethyl bromoacetate is removed under vacuum to result in 21 g of phosphonate in the form of a white solid.

¹H NMR: 1.08 (t, J=7.15 Hz, 3H), 1.30 (s, 18H), 3.29 (d, J=21.7 Hz, 2H), 4.05 (q, J=7.15 Hz, 2H), 7.02-7.07 (m, 4H), 7.29 (dt, J=7.7 Hz, J=1.6 Hz, 2H), 7.61 (dt, J=7.9 Hz, J=1.1 Hz, 2H)

³¹P NMR: 10.4 ppm

### Example A4

Second Route For the Synthesis of the Phosphonate III

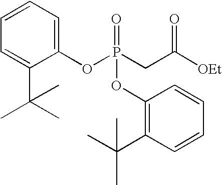

300 ml of toluene, 18.9 g of PCl₃ (0.14 mmol) and 39.8 g of 2-(tert-butyl)phenol (0.27mmol) are stirred and cooled to −10° C. 59 g of tripropylamine (0.41 mmol) are subsequently run in over approximately 2 h, which makes it possible to maintain a temperature of the order of −5° C. After maintaining for 1 h, 5.9 g of absolute ethanol (0.13 mmol) are added over 30 minutes and then the medium is left stirring at ambient temperature overnight before treatment. The organic phase is then washed with water and then treated over basic alumina in order to remove possible phosphorus-comprising byproducts. The solvent is subsequently evaporated to result in 42 g of mixed phosphite. 20 g (53 mmol) of this phosphite are subsequently added over 1 h to 16.3 g (106 mmol) of ethyl bromoacetate at 130° C. After reacting for 20 h, the excess ethyl bromoacetate is removed under vacuum to result in 21 g of phosphonate in the form of a white solid.

¹H NMR: 1.08 (t, J=7.15 Hz, 3H), 1.30 (s, 18H), 3.29 (d, J=21.7 Hz, 2H), 4.05 (q, J=7.15 Hz, 2H), 7.02-7.07 (m, 4H), 7.29 (dt, J=7.7 Hz, J=1.6 Hz, 2H), 7.61 (dt, J=7.9 Hz, J=1.1 Hz, 2H)

³¹P NMR: 10.4 ppm

### Example B

### Test Results of the Phosphonates of the Invention In the Horner-Wadsworth-Emmons Reaction

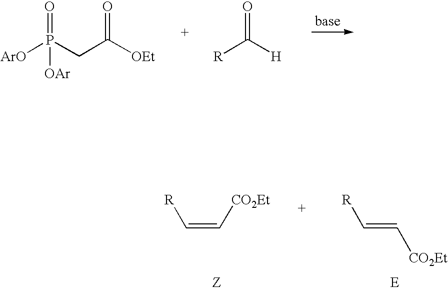

The HWE reactions presented as examples are analyzed by gas chromatography using a Varian Star 3400CX device. The column used is a DB1 125-1034 from J&W Scientific (length: 30 m, internal diameter: 0.53 mm and film thickness of 3 μm). The starting temperature of the column is 100° C. and the rise in temperature is 7° C. per minute. Under these conditions, the retention times of the various compounds are summarized in the following table:

TABLE I

| | t_{R} |
|---|---|
| Compound | (min) |
| 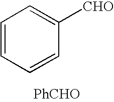 | 4.8 |
| 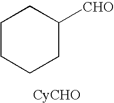 | 4.5 |
| n-C₇H₁₅CHO | 5.3 |
| 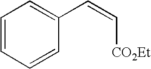 | 11.8 |
| 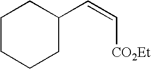 | 10.8 |
| 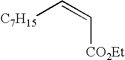 | 12.1 |
| 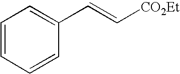 | 13.5 |
| 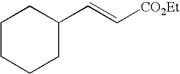 | 12.5 |
| 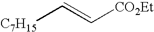 | 13.1 |

The diastereoselectivity factor S (S=Z/(Z+E) in %) is defined by the area ratio of the amount of Z isomer to the sum of the Z and E isomers formed.

The Z and E isomers are defined in the framed reaction scheme on the preceding page. The conversion (Conv=(Z+E)/(Z+E+phosphonate) in %) is also defined by the area ratio of the amount of olefin formed to the sum of the amounts of olefin formed and of residual phosphonate.

### Example B1

NaI/TMG Or NaI/DBU

Procedure

0.5 mmol of phosphonate (1.1 eq) and 0.6 mmol of NaI (1.3 eq) are dissolved in 10 ml of THF. The mixture is then cooled to 0° C. before the addition of 0.55 mmol (1.2 eq) of tetramethylguanidine (TMG) or of diazabicycloundecene (DBU). After approximately thirty minutes, the reaction medium is brought to the desired temperature in order to carry out the conversion. After stabilizing the temperature, 0.45 mmol of aldehyde (1 eq) is added. The reaction is then monitored by treatment of an aliquot with a saturated ammonium chloride solution and extraction of the mixture with toluene.

In the examples below, the value obtained with a reference phosphonate described by Ando, K., Oishi, T., Hirama, M., Ohno, H. and Ibuka, T, J. Org. Chem., 2000, 65, 4745-4749, has been added between brackets in the selectivity column.

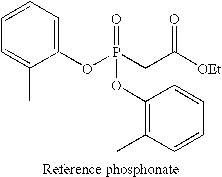

This is the phosphonate prepared from ortho-cresol.

TABLE II

| | Phos- | Aldehyde | | | |
|---|---|---|---|---|---|
| Example | phonate | (R₄) | Conditions | S (Ref) | Conv |
| B1.1 | I | Ph | TMG/−78° C./3 h | 95 (82) | 97 |
| B1.2 | I | Ph | TMG/0° C./1 h | 83 (69) | 98 |
| B1.3 | I | Cy | TMG/−78° C./3 h | 95 (95) | 91 |
| B1.4 | I | Cy | TMG/0° C./1 h | 91 (89) | 97 |
| B1.5 | I | n-C₇H₁₅ | TMG/−78° C./3 h | 96 (93) | 90 |
| B1.6 | I | n-C₇H₁₅ | TMG/0° C./1 h | 89 (85) | 95 |
| B1.7 | II | Ph | TMG/0° C./1 h | 81 (69) | 95 |
| B1.8 | II | Cy | TMG/0° C./1 h | 94 (89) | 93 |
| B1.9 | II | n-C₇H₁₅ | TMG/0° C./1 h | 91 (85) | 95 |
| B1.10 | III | Ph | TMG/−78° C./24 h | 95 (82) | 100 |
| B1.11 | III | Ph | TMG/0° C./1 h | 81 (69) | 100 |
| B1.12 | III | Cy | TMG/−78° C./24 h | 98 (95) | 100 |
| B1.13 | III | Cy | TMG/0° C./1 h | 95 (89) | 100 |
| B1.14 | III | Cy | TMG, 0.2 eq NaI/ | 94 | 75 |
| | | | 0° C./2 h | | |
| B1.15 | III | Cy | DBU, 0.2 eq NaI/ | 95 | 90 |
| | | | 0° C./1 h | | |
| B1.16 | III | n-C₇H₁₅ | TMG/−78° C./4 h | 98 (93) | 94 |
| B1.17 | III | nC₇H₁₅ | TMG/0° C./1 h | 92 (85) | 100 |

It may be observed that the phosphonates I, II and III always result in selectivities at least equal to the reference phosphonate under identical conditions. Regarding the phosphonates II and III more particularly, the selectivities obtained at 0° C. are even very close to those obtained with the reference phosphonate at −78° C., which represents an increase of nearly 80° C. for the same Z/E ratio of olefins.

The examples which follow show that high selectivities are obtained at 0° C. and even at ambient temperature under various conditions of base and of solvent.

### Example B2

NaHMDS Or KHMDS

Procedure

0.5 mmol of phosphonate is dissolved in 10 ml of THF. The solution is then cooled to 0° C. before the addition of 0.45 mmol of NaHMDS or KHMDS. After approximately 10 minutes, 0.45 mmol of aldehyde is added. The reaction is then monitored by treatment of an aliquot with a saturated ammonium chloride solution and extraction of the mixture with toluene.

TABLE III

| | Phos- | Aldehyde | | | |
|---|---|---|---|---|---|
| Example | phonate | (R₄) | Conditions | S | Conv |
| B2.1 | III | Ph | KHMDS/1 h | 93 | 100 |
| B2.2 | III | Ph | NaHMDS/1 h | 83 | 100 |
| B2.3 | III | Cy | KHMDS/1 h | 94 | 97 |
| B2.4 | III | Cy | NaHMDS/1 h | 95 | 97 |
| B2.5 | III | n-C₇H₁₅ | KHMDS/1 h | 93 | 98 |
| B2.6 | III | n-C₇H₁₅ | NaHMDS/1 h | 93 | 99 |

### Example B3

tBuOK

Procedure

0.5 mmol of phosphonate is dissolved in 10 ml of THF. The solution is then cooled to 0° C. before the addition of 0.45 mmol of tBuOK. After approximately 10 minutes, 0.45 mmol of aldehyde is added. The reaction is then monitored by treatment of an aliquot with a saturated ammonium chloride solution and extraction of the mixture with toluene.

TABLE IV

| | Phos- | Aldehyde | | | |
|---|---|---|---|---|---|
| Example | phonate | (R₄) | Conditions | S | Conv |
| B3.1 | III | Ph | tBuOK/1 h | 93 | 70 |
| B3.2 | III | Cy | tBuOK/1 h | 94 | 70 |
| B3.3 | III | n-C₇H₁₅ | tBuOK/1 h | 94 | 75 |

### Example B4

K₂CO₃ Or Cs₂CO₃

Procedure

0.5 mmol of phosphonate and 1 mmol of carbonate are diluted in 10 ml of solvent. The solution is then cooled at 0° C. for 30 minutes before the addition of 0.45 mmol of aldehyde. The reaction is then monitored by treatment of an aliquot with a saturated ammonium chloride solution and extraction of the mixture with toluene.

TABLE V

| | Phos- | Aldehyde | | | |
|---|---|---|---|---|---|
| Example | phonate | (R₄) | Conditions | S | Conv |
| B4.1 | III | Ph | K₂CO₃/NMP/72 h | 82 | 65 |
| B4.2 | III | Ph | K₂CO₃/DMAC/72 h | 84 | 80 |
| B4.3 | III | Ph | K₂CO₃/DMF/54 h | 87 | 98 |
| B4.4 | III | Ph | K₂CO₃/THF/54 h | 89 | 88 |
| B4.5 | III | Ph | K₂CO₃/CH₃CN/54 h | 93 | 90 |
| B4.6 | III | Ph | Cs₂CO₃/NMP/96 h | 74 | 100 |
| B4.7 | III | Ph | Cs₂CO₃/DMAC/96 h | 75 | 100 |
| B4.8 | III | Ph | Cs₂CO₃/DMF/96 h | 78 | 100 |
| B4.9 | III | Ph | Cs₂CO₃/THF/96 h | 91 | 100 |
| B4.10 | III | Ph | Cs₂CO₃/CH₃CN/1 h | 91 | 100 |

### Example B5

NaOH Or KOH

Procedure

0.5 mmol of phosphonate and 1 mmol of base are diluted in 10 ml of THF and cooled to 0° C. The aldehyde (0.45 mmol) is then added and the reaction is monitored by treatment of an aliquot with a saturated ammonium chloride solution and extraction of the mixture with toluene.

TABLE VI

| | Phos- | Aldehyde | | | |
|---|---|---|---|---|---|
| Example | phonate | (R₄) | Conditions | S | Conv |
| B5.1 | III | Ph | KOH/1 h | 93 | 100 |
| B5.2 | III | Cy | KOH/1 h | 95 | 100 |
| B5.3 | III | n-C₇H₁₅ | KOH/1 h | 93 | 100 |
| B5.4 | III | Ph | NaOH/1 h | 86 | 98 |
| B5.5 | III | Cy | NaOH/1 h | 95 | 98 |
| B5.6 | III | n-C₇H₁₅ | NaOH/1 h | 93 | 98 |

### Example B6

K₃PO₄

Procedure

0.5 mmol of phosphonate and 1 mmol of K₃PO₄ are diluted in 10 ml of solvent. The solution is then stirred at 22° C. for 30 minutes before the addition of 0.45 mmol of aldehyde. The reaction is then monitored by treatment of an aliquot with a saturated ammonium chloride solution and extraction of the mixture with toluene.

TABLE VII

| | Phos- | Aldehyde | | | |
|---|---|---|---|---|---|
| Example | phonate | (R₄) | Conditions | S | Conv |
| B6.1 | III | Ph | CH₃CN/2 h | 92 | 94 |
| B6.2 | III | Cy | CH₃CN/4 h | 92 | 91 |
| B6.3 | III | n-C₇H₁₅ | CH₃CN/4 h | 91 | 94 |
| B6.4 | III | Ph | THF/20 h | 88 | 88 |
| B6.5 | III | Cy | THF/20 h | 92 | 77 |
| B6.6 | III | n-C₇H₁₅ | THF/20 h | 90 | 94 |
| B6.7 | III | Ph | DMF/1 h | 86 | 100 |
| B6.8 | III | Cy | DMF/2 h | 84 | 92 |
| B6.9 | III | n-C₇H₁₅ | DMF/1 h | 85 | 97 |
| B6.10 | III | Ph | MGN/4 h | 89 | 85 |
| B6.11 | III | Cy | MGN/72 h | 91 | 100 |
| B6.12 | III | n-C₇H₁₅ | MGN/72 h | 87 | 100 |

1-21. (canceled)

22. A process for the diastereoselective preparation of olefins (C) by the Horner-Wadsworth-Emmons reaction comprising the step of reacting a phosphonate (A) with a carbonyl derivative (B) in the presence of a base in an appropriate solvent, 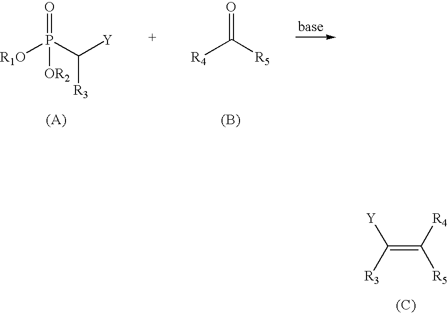
in which the compounds (A), (B) and (C) are such that:
Y represents an electron-withdrawing group selected from the group consisting of:
CO₂R,
CN,
C(O)R,
S(O)R,
S(O)₂R,
C(O)NRR′,
N═CRR′, and
P(O)OROR′,
with R and R′ as defined below,
R₅, R and R′, taken independently, are identical or different and they represent:
a hydrogen atom;
a saturated or unsaturated and linear or branched aliphatic radical having from 1 to 24 carbon atoms which is optionally substituted by heteroatoms;
a saturated, unsaturated or aromatic and monocyclic or polycyclic cycloaliphatic radical having from 4 to 24 carbon atoms which is optionally substituted by heteroatoms; or
a saturated or unsaturated and linear or branched aliphatic radical carrying a cyclic substituent which is optionally substituted by heteroatoms in the aliphatic part and/or the cyclic part;
R and R′ optionally form together a saturated, unsaturated or aromatic ring optionally comprising heteroatoms;
R₃ represents a radical selected from the group consisting of:
R,
a halogen atom,
OR,
SR,
NRR′, and
with R and R′ as defined above,
R₄ represents a radical selected from the group consisting of:
a saturated or unsaturated and linear or branched aliphatic radical having from 1 to 24 carbon atoms which is optionally substituted by heteroatoms;
a saturated, unsaturated or aromatic and monocyclic or polycyclic cycloaliphatic radical having from 4 to 24 carbon atoms which is optionally substituted by heteroatoms; it being possible for the heteroatoms also to be present in the cyclic part; and
a saturated or unsaturated and linear or branched aliphatic radical carrying a cyclic substituent which is optionally substituted by heteroatoms in the aliphatic part and/or the cyclic part;
with the further proviso that R₄ has priority over R₅ according to the Cahn-Ingold-Prelog rules,
wherein R₁ and R₂, taken independently, are identical or different and they represent a radical of formula (I): 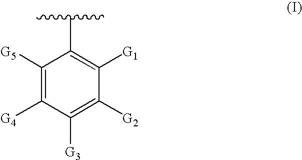
in which:
G₁, G₂, G₃, G₄ and G₅, taken independently, are identical or different and they represent:
a hydrogen atom,
an alkyl radical having from 1 to 24 carbon atoms, being:
a saturated or unsaturated and linear or branched aliphatic radical which is optionally substituted by heteroatoms; such as, for example, a carbon atom bonded to three carbon atoms, and preferably tert-butyl;
a saturated, unsaturated or aromatic and monocyclic or polycyclic cycloaliphatic radical having from 4 to 24 carbon atoms which is optionally substituted by:
an alkoxy radical having from 1 to 24 carbon atoms,
a halogen atom,
an oxygen atom, a sulfur atom or a nitrogen atom, it being possible for the heteroatom also to be present in the cyclic part; or
a saturated or unsaturated and linear or branched aliphatic radical carrying a cyclic substituent which is optionally substituted by heteroatoms in the aliphatic part and/or the cyclic part;
an alkoxy radical having from 1 to 24 carbon atoms,
a halogen atom, or
a heteroatom, such as an oxygen atom, a sulfur atom or a nitrogen atom,
optionally G₁, G₂, G₃, G₄ or G₅ together forming, between two neighboring groups, a saturated, unsaturated or aromatic ring having from 4 to 6 carbon atoms and optionally comprising heteroatoms,
with the further proviso that at least one of the G₁ or G₅ radicals is taken independently and represents a radical formed by a carbon atom itself connected to three carbon atoms, and optionally a tert-butyl radical, or a phenyl radical optionally substituted by one or more radicals chosen from alkoxy radicals having from 1 to 24 carbon atoms, halogen atoms or heteroatoms.

23. The process as claimed in claim 22, wherein the phosphonate (A) comprises identical or different R₁ and R₂ groups having the formula (I) in which at least one of the G₁ or G₅ radicals is taken independently and represents a radical formed by a carbon atom itself connected to three carbon atoms, and optionally a tert-butyl radical.

24. The process as claimed in claim 22, wherein the phosphonate is of formula (A) in which R₁ is identical to R₂ and has the formula (I) in which:
G₁ is tert-butyl and G₂, G₃, G₄ and G₅ are hydrogen atoms,
G₁ and G₃ are tert-butyl radicals and G₂, G₄ and G₅ are hydrogen atoms, or
G₁ is a phenyl radical and G₂, G₃, G₄ and G₅ are hydrogen atoms.

25. The process as claimed in claim 24, wherein Y represents CO₂R, with R representing a hydrogen atom or a saturated or unsaturated and linear, branched or cyclic alkyl radical having from 1 to 12 carbon atoms,
and R₃ represents a hydrogen atom.

26. The process as claimed in claim 25, wherein Y represents a CO₂R radical, with R representing an ethyl radical, and R₃ represents a hydrogen atom.

27. The process as claimed in claim 22, wherein the carbonyl derivative used for the reaction is an aldehydes, with R₅ representing a hydrogen atom.

28. The process as claimed in claim 27, wherein the aldehyde used is such that R₄ is an aliphatic radical and optionally comprises ethylenic unsaturations.

29. The process as claimed in claim 28, wherein the R₄ radical is cyclohexyl.

30. The process as claimed in claim 27, wherein the R₄ radical used is aromatic and optionally comprises one or more substitutions by alkoxy groups having from 1 to 6 carbon atoms or halogen atoms or CF₃ groups.

31. The process as claimed in claim 30, wherein the R₄ radical is a phenyl radical.

32. The process as claimed in claim 22, wherein the base is selected from the group consisting of:
amides of formula MNR″R″′ with M an alkali metal, and R″ and R″′ are alkyl radicals or alkylsilane radicals,
alkoxides of formula MOR″ with M an alkali metal, and R″ being alkyl radicals,
hydrides of formula MH with M an alkali metal,
carbonates of formula M₂CO₃ or MCO₃ with M an alkali metal, or an alkaline earth,
alkali metal or alkaline earth metal hydroxides,
alkali metal or alkaline earth metal phosphates, and
organic nitrogenous bases of amine, amidine or guanidine, optionally in combination with alkali metal or alkaline earth metal halides.

33. The process as claimed in claim 32, wherein the base is selected from the group consisting of:
alkoxides of MOR″ formula with M an alkali metal, and R″ being alkyl radicals, -carbonates of M₂CO₃ or MCO₃ formula with M an alkali metal, or an alkaline earth metal
alkali metal or alkaline earth metal hydroxides,
alkali metal or alkaline earth metal phosphates, or
organic nitrogenous bases of amine, amidine or guanidine, optionally in combination with alkali metal or alkaline earth metal halides.

34. The process as claimed in claim 32, wherein the base is:
carbonates of M₂CO₃ or MCO₃ formulae with M an alkali metal, or an alkaline earth
metal,
alkali metal or alkaline earth metal hydroxides, or
alkali metal or alkaline earth metal phosphates.

35. The process as claimed in claim 22, wherein the solvent used is an ether, optionally tetrahydrofuran (THF) or dioxane.

36. The process as claimed in claim 22, wherein the solvent used are nitriles having from 1 to 8 carbon atoms, optionally acetonitrile.

37. The process as claimed in in claim 22, wherein the solvent used is a polar amide solvent, optionally dimethylformamide (DMF), N-methylpyrrolidone (NMP) or dimthylacetamide (DMAC).

38. The process as claimed in claim 35, wherein the amount of solvent used is between 0.5 ml and 20 ml per mmol of phosphonate (A).

39. The process as claimed in claim 22, carried out at a temperature maintained at a temperature of between −100° C. and +100° C.

40. The process as claimed in claim 22, wherein the temperature is maintained at a temperature of between −50° C. and +50° C.

41. The process as claimed in claim 22, wherein the temperature is maintained at a temperature of between −20° C. and +50° C., optionally of of between −10° C. and +25° C.
